SEQUENCE LISTING (30 sequences)

SEQ ID NO: 1 - RNA, 24 nt, synthetic construct
  source 1..24: mol_type = other RNA
tggaagactagtgattttgttgtt

SEQ ID NO: 2 - RNA, 22 nt, synthetic construct
  source 1..22: mol_type = other RNA
caacaaatcacagtctgccata

SEQ ID NO: 3 - RNA, 22 nt, synthetic construct
  source 1..22: mol_type = other RNA
  misc_difference >22: note = (dT)5
caacaaatcacagtctgccata

SEQ ID NO: 4 - RNA, 22 nt, synthetic construct
  source 1..22: mol_type = other RNA
  misc_difference >22: note = (dC)5
caacaaatcacagtctgccata

SEQ ID NO: 5 - RNA, 22 nt, synthetic construct
  source 1..22: mol_type = other RNA
  misc_difference >22: note = (dA)5
caacaaatcacagtctgccata

SEQ ID NO: 6 - RNA, 22 nt, synthetic construct
  source 1..22: mol_type = other RNA
  misc_difference >22: note = (dG)5
caacaaatcacagtctgccata

SEQ ID NO: 7 - RNA, 22 nt, synthetic construct
  source 1..22: mol_type = other RNA
ttgcatagtcacaaaagtgatc

SEQ ID NO: 8 - RNA, 22 nt, synthetic construct
  source 1..22: mol_type = other RNA
tcatttttgtgatgttgcagct

SEQ ID NO: 9 - RNA, 22 nt, synthetic construct
  source 1..22: mol_type = other RNA
  misc_difference >22: note = (dT)5
tcatttttgtgatgttgcagct

SEQ ID NO: 10 - RNA, 22 nt, synthetic construct
  source 1..22: mol_type = other RNA
  misc_difference >22: note = (dC)5
tcatttttgtgatgttgcagct

SEQ ID NO: 11 - RNA, 22 nt, synthetic construct
  source 1..22: mol_type = other RNA
  misc_difference >22: note = (dA)5
tcatttttgtgatgttgcagct

SEQ ID NO: 12 - RNA, 22 nt, synthetic construct
  source 1..22: mol_type = other RNA
  misc_difference >22: note = (dG)5
tcatttttgtgatgttgcagct

SEQ ID NO: 13 - RNA, 22 nt, synthetic construct
  source 1..22: mol_type = other RNA
caatcactaactccactgccat

SEQ ID NO: 14 - RNA, 23 nt, synthetic construct
  source 1..23: mol_type = other RNA
taggcagtgtcattagctgattg

SEQ ID NO: 15 - RNA, 23 nt, synthetic construct
  source 1..23: mol_type = other RNA
  misc_difference >23: note = (dT)5
taggcagtgtcattagctgattg

SEQ ID NO: 16 - RNA, 23 nt, synthetic construct
  source 1..23: mol_type = other RNA
  misc_difference >23: note = (dC)5
taggcagtgtcattagctgattg

SEQ ID NO: 17 - RNA, 23 nt, synthetic construct
  source 1..23: mol_type = other RNA
  misc_difference >23: note = (dA)5
taggcagtgtcattagctgattg

SEQ ID NO: 18 - RNA, 23 nt, synthetic construct
  source 1..23: mol_type = other RNA
  misc_difference >23: note = (dG)5
taggcagtgtcattagctgattg

SEQ ID NO: 19 - RNA, 23 nt, synthetic construct
  source 1..23: mol_type = other RNA
aggcagtgtagttagctgattgc

SEQ ID NO: 20 - RNA, 22 nt, synthetic construct
  source 1..22: mol_type = other RNA
aatcactaaccacacggccagg

SEQ ID NO: 21 - RNA, 22 nt, synthetic construct
  source 1..22: mol_type = other RNA
  misc_difference >22: note = (dT)5
aatcactaaccacacggccagg

SEQ ID NO: 22 - RNA, 22 nt, synthetic construct
  source 1..22: mol_type = other RNA
  misc_difference >22: note = (dC)5
aatcactaaccacacggccagg

SEQ ID NO: 23 - RNA, 22 nt, synthetic construct
  source 1..22: mol_type = other RNA
  misc_difference >22: note = (dA)5
aatcactaaccacacggccagg

SEQ ID NO: 24 - RNA, 22 nt, synthetic construct
  source 1..22: mol_type = other RNA
  misc_difference >22: note = (dG)5
aatcactaaccacacggccagg

SEQ ID NO: 25 - RNA, 24 nt, synthetic construct
  source 1..24: mol_type = other RNA
ttaatgctaatcgtgataggggtt

SEQ ID NO: 26 - RNA, 22 nt, synthetic construct
  source 1..22: mol_type = other RNA
ctcctacatattagcattaaca

SEQ ID NO: 27 - RNA, 22 nt, synthetic construct
  source 1..22: mol_type = other RNA
  misc_difference >22: note = (dT)5
ctcctacatattagcattaaca

SEQ ID NO: 28 - RNA, 22 nt, synthetic construct
  source 1..22: mol_type = other RNA
  misc_difference >22: note = (dC)5
ctcctacatattagcattaaca

SEQ ID NO: 29 - RNA, 22 nt, synthetic construct
  source 1..22: mol_type = other RNA
  misc_difference >22: note = (dA)5
ctcctacatattagcattaaca

SEQ ID NO: 30 - RNA, 22 nt, synthetic construct
  source 1..22: mol_type = other RNA
  misc_difference >22: note = (dG)5
ctcctacatattagcattaaca

(19) United States
(12) Patent Application Publication
(10) Pub. No.: US 2024/0132891 A1
(43) Pub. Date: Apr. 25, 2024

(54) MICRORNA COMPOSITIONS AND METHODS OF USE THEREOF FOR THE TREATMENT OF NERVOUS SYSTEM DYSFUNCTION

(71) Applicant: Rutgers, The State University of New Jersey, New Brunswick, NJ (US)

(72) Inventors: M. Maral Mouradian, Princeton, NJ (US); Eunsung Junn, East Brunswick, NJ (US)

(73) Assignee: Rutgers, The State University of New Jersey, New Brunswick, NJ (US)

(21) Appl. No.: 18/484,001

(22) Filed: Oct. 9, 2023

Related U.S. Application Data
(60) Provisional application No. 63/380,520, filed on Oct. 21, 2022.

(51) Int. Cl. C12N 15/113; A61K 45/06; C12N 15/86

(52) U.S. Cl. CPC C12N 15/113; A61K 45/06; C12N 15/86; C12N 2310/141; C12N 2750/14141

(57) ABSTRACT

MicroRNAs, or mimics thereof, for treating a subject suffering from nervous system dysfunction are described. Pharmaceutical compositions, viral vectors or viral particles, and nanoparticles comprising these microRNAs, or mimics thereof, are described. Methods of using these microRNAs, or mimics thereof, pharmaceutical compositions comprising the microRNAs, viral vectors, or nanoparticles are also described herein. These compositions and methods of use will provide novel therapies for patients suffering nervous system dysfunction, including but not limited to alpha-synucleinopathies and SCI.

### CROSS-REFERENCE TO RELATED APPLICATIONS

This application claims priority under 35 U.S.C. § 119(e) to U.S. Provisional Patent Application No. 63/380,520, filed Oct. 21, 2022. The foregoing application is incorporated by reference herein in its entirety.

### STATEMENT REGARDING FEDERALLY SPONSORED RESEARCH

This invention was made with government support under grant number NS070898 awarded by the National Institutes of Health. The United States government has certain rights in the invention.

### REFERENCE TO A SEQUENCE LISTING

This application contains a Sequence Listing which has been submitted electronically in xml format and is hereby incorporated by reference in its entirety. Said xml copy, created on Nov. 7, 2023, is named “Sequence Listing Sequence listing 09673800761.xml” and is 35,077 bytes in size.

### FIELD OF THE INVENTION

The invention relates generally to the fields of medicine, molecular biology, and gene therapy. In particular, the invention relates to compositions, vectors, viruses, nanoparticles, kits, and methods for delivering microRNA for therapeutic use in nervous system dysfunction.

### BACKGROUND OF THE INVENTION

Nervous system dysfunction can result in severe symptoms as a consequence of several different types of injury. For example, neurodegenerative disorders (NDs) are a group of related human maladies that share a common pathophysiological feature, the progressive degeneration of selective neuronal populations over the course of time. Despite significant progress in elucidating the genetic causes underlying these disparate disorders, relatively little is known about the biochemical mechanisms that cause the selective neuronal degeneration common to all of them.

One common neurodegenerative disorder is Parkinson's disease (PD), which affects 1% of the population over 65. It is characterized by disabling motor abnormalities including tremor, slow movements, rigidity and poor balance. These impairments stem from the progressive loss of dopaminergic neurons in the substantia nigra pars compacta. Eventually, large percentages of patients develop dementia and hallucinations when the pathology involves other brain regions as well, leading to Parkinson's Disease Dementia (PDD). Although the majority of Parkinson cases appear to be sporadic, the disorder runs in families in about 15-20% of the cases. To date, several distinct genes have been identified to cause PD including α-synuclein, parkin, dj-1, pink1 and lrrk2, and several additional risk genes increase the chance of developing the disease. Understanding how mutations in these genes cause neurodegeneration is crucial in the development of new treatments that might slow or stop the disease progression. Thus, there remains a need for new treatments for the disease progression and for assays to help identify agents for such treatment.

In another cause of nervous system dysfunction, spinal cord injury (SCI) involves damage to the spinal cord including nerves within the bony protection of the spine due to trauma or disease. SCI could result in loss of muscle function, sensation, or autonomic function in the parts of the body served by the spinal cord below the level of the injury. These changes in the functionality could be temporary or permanent depending on the severity, type, size, and site/location of injury. According to the National Spinal Cord Injury Association, as many as 450,000 people in the United States are living with SCI. Every year, an estimated 17,000 new SCIs occur in the U.S, and as per the Centers for Disease Control and Prevention (CDC), SCI costs the nation an estimated $9.7 billion each year. There is a great need to find a treatment for SCI.

Accumulating evidence indicates that increased levels of certain proteins, including the protein α-synuclein, are deleterious to neurons and can lead to neurodegenerative disorders, including Parkinson's disease, Parkinson's disease Dementia, and Dementia with Lewy Bodies (DLB). Similar increases in other protein levels, such as VDAC1, have also been associated with increased oxidative stress in SCI and other disease processes resulting in nervous system dysfunction. With the increasing number of individuals affected by nervous system dysfunction of various causes and the increasing costs associated with caring for these individuals, there is a dire need for novel therapies that prevent and treat these conditions.

### SUMMARY OF THE INVENTION

This disclosure provides, in a first aspect, microRNA, or a mimic thereof, for the therapeutic treatment of nervous system dysfunction in a patient in need thereof. In this aspect, the microRNA, or mimic thereof, comprises a double-stranded oligonucleotide RNA structure, further comprising a first guide RNA nucleotide strand and a second passenger RNA nucleotide strand.

In another set of embodiments, the microRNA, or mimic thereof, may contain one or more chemical or nonchemical modifications. In certain embodiments, these modifications may be at the 5′ end, 3′ end, or both, of one or more of the RNA nucleotide strands. In certain embodiments, these modifications may comprise the addition of one or more deoxyribonucleotides, selected from deoxythymidine (dT), deoxyadenosine (dA), deoxycytidine (dC), and deoxyguanosine (dG). In preferred embodiments, five deoxyribonucleotides are added to one or more of the RNA strands. In preferred embodiments, the modification, including the one or more deoxyribonucleotides, are added to the 3′ end of the passenger RNA strand.

In yet another set of embodiments, the double-strand miRNA comprises a microRNA selected from the group comprising miRNA-7, miRNA-153, miRNA-34b, miRNA-34c, and miRNA-155.

In certain embodiments, the microRNA, or mimic thereof, comprises a first guide nucleotide sequence having at least 90% identity to SEQ ID NO: 1 or comprising SEQ ID NO: 1.

In certain embodiments, the microRNA, or mimic thereof, comprises a second passenger sequence having at least 90% identity to a nucleic acid sequence selected from SEQ ID NOs: 2-6 or comprising a nucleotide sequence selected from SEQ ID NOs: 2-6.

In certain embodiments, the microRNA, or mimic thereof, comprises a first guide nucleotide sequence having at least 90% identity to SEQ ID NO: 7 or comprising SEQ ID NO: 7.

In certain embodiments, the microRNA, or mimic thereof, comprises a second passenger nucleotide sequence having at least 90% identity to a nucleic acid sequence selected from SEQ ID NOs: 8-12 or comprising a nucleotide sequence selected from SEQ ID NOs: 8-12.

In certain embodiments, the microRNA, or mimic thereof, comprises a first guide nucleotide sequence having at least 90% identity to SEQ ID NO: 13 or comprising SEQ ID NO: 13.

In certain embodiments, the microRNA, or mimic thereof, comprises a second passenger nucleotide sequence having at least 90% identity to a nucleic acid sequence selected from SEQ ID NOs: 14-18 or comprising a nucleotide sequence selected from SEQ ID NOs: 14-18.

In certain embodiments, the microRNA, or mimic thereof, comprises a first guide nucleotide sequence having at least 90% identity to SEQ ID NO: 19 or comprising SEQ ID NO: 19.

In certain embodiments, the microRNA, or mimic thereof, comprises a second passenger nucleotide sequence having at least 90% identity to a nucleic acid sequence selected from SEQ ID NOs: 20-24 or comprising a nucleotide sequence selected from SEQ ID NOs: 20-24.

In certain embodiments, the microRNA, or mimic thereof, comprises a first guide nucleotide sequence having at least 90% identity to SEQ ID NO: 25 or comprising SEQ ID NO: 25.

In certain embodiments, the microRNA, or mimic thereof, comprises a second passenger nucleotide sequence having at least 90% identity to a nucleic acid sequence selected from SEQ ID NOs: 26-30 or comprising a nucleotide sequence selected from SEQ ID NOs: 26-30.

In certain embodiments, the disclosure provides a pharmaceutical composition comprising microRNA, or mimic thereof, for the treatment of nervous system dysfunction.

In certain embodiments, the disclosure provides a viral vector or viral particle comprising the miRNA, or mimic thereof, for the treatment of nervous system dysfunction, or a nucleic acid sequence encoding the microRNA or mimic thereof for the treatment of nervous system dysfunction.

In certain embodiments, the disclosure provides a nanoparticle comprising the microRNA, or mimic thereof, for the treatment of nervous system dysfunction, or the vector or viral particle comprising the miRNA, or mimic thereof, for the treatment of nervous system dysfunction.

In a second aspect, the present disclosure provides a method for the treatment of nervous system dysfunction in a patient in need thereof by administering an effective amount of a composition comprising miRNA, or a mimic thereof. In certain embodiments, the method comprises administering a pharmaceutical composition comprising the microRNA, or mimic thereof. In certain embodiments, the method may comprise administering the vector or viral particle comprising the miRNA, or a mimic thereof. In certain embodiments, the method may comprise administering the nanoparticle comprising the miRNA, or mimic thereof.

In certain embodiments, the method comprises administering the miRNA, or mimic thereof, the pharmaceutical composition, the vector or viral particle, or the nanoparticle to the patient topically, by inhalation, intradermally, transdermally, parenterally, intravenously, intramuscularly, intranasally, subcutaneously, percutaneously, intratracheally, intraventricularly, intra-brain tissue, intra-spinal cord, intraperitoneally, intra-tumorally, by perfusion, by lavage, orally, or by direct injection.

In certain embodiments, the method comprises administering the microRNA, or mimic thereof, to the patient in one or more doses of 0.01 mg/kg to 10 g/kg per body weight of the patient, or administering the vector or viral particle to the patient in one or more doses of 1×10¹⁰ to 5×10¹⁴ viral genomes/ml.

In certain non-limiting embodiments, the nervous system dysfunction may result from a neurodegenerative disease. In certain embodiments, the neurodegenerative disease may be associated with alpha-synuclein pathology. In certain embodiments, the alpha-synuclein pathology may be Parkinson's Disease, Dementia with Lewy Bodies, Parkinson's Disease Dementia, or Multiple System Atrophy.

In different non-limiting embodiments, the nervous system dysfunction may result from SCI, TBI, Multiple Sclerosis, Optic Neuritis, Transverse Myelitis, Neuromyelitis Optica (NMO), Acute Disseminated Encephalomyelitis, Ischemic Stroke, Amyotrophic Lateral Sclerosis, and Autism Spectrum Disorders (ASD).

In a third aspect, the disclosure provides a method for the treatment of nervous system dysfunction in a patient in need thereof, comprising administering a second agent to the patient. In certain embodiments, the second agent comprises any of the miRNA, or mimic thereof, pharmaceutical compositions, viral vector or viral particle, or nanoparticles described herein.

### DETAILED DESCRIPTION OF THE INVENTION

In general, this disclosure is drawn to novel compositions of microRNA and methods for treating nervous system dysfunction utilizing these microRNA compositions.

Described herein are compositions comprising a therapeutically effective amount of a miRNA, or mimic thereof, with enhanced strand selectivity for improving nervous system function in a subject (e.g., human). Methods of using these compositions are also described herein.

As used herein, the phrase “effective amount” means an amount for providing the therapeutic effect of the composition or substance being administered.

As used herein, the term “mimic” refers to a composition which, although structurally different, exhibits the same mechanism of action or effect of another composition or substance, thereby producing the same or essentially the same end result.

As used herein, the term “patient” refers to a mammal, human or otherwise, suffering from a disease or condition.

As used herein, the term “subject” refers to organisms to be treated by the methods of the present disclosure. Such organisms can be any type of organism, e.g., single-cell organisms to more complex organisms such as eukaryotes (e.g., rodents, bovines, porcines, canines, felines, and the like). For the purposes of this application, the term “subject” also includes any substance derived from an organism, for example, a subject may be cellular tissue derived from a mammal used in in-vitro testing.

As used herein the term “treating” includes abrogating, substantially inhibiting, slowing, or reversing the progression of a condition, substantially ameliorating clinical or aesthetical symptoms of a condition, substantially preventing the appearance of clinical or aesthetical symptoms of a condition, and protecting from harmful or annoying stimuli.

It must be noted that as used herein and in the appended claims, the singular forms “a”, “and”, and “the” include plural references unless the context clearly dictates otherwise.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As used herein, the term “RNA” includes a molecule comprising at least one ribonucleotide residue.

As used herein, the term “gene” includes a nucleic acid molecule that codes for a particular protein, or in certain cases, a functional or structural RNA (ribonucleic acid) molecule.

As used herein, a “nucleic acid” or a “nucleic acid molecule” means a chain of two or more nucleotides such as RNA and DNA (deoxyribonucleic acid).

As used herein, the phrase “expression control sequence” refers to a nucleic acid that regulates the replication, transcription, and translation of a coding sequence in a recipient cell. Examples of expression control sequences include promoter sequences, polyadenylation (pA) signals, introns, transcription termination sequences, enhancers, silencer, upstream regulatory domains, origins of replication, and internal ribosome entry sites (“IRES”).

When referring to a nucleic acid molecule or polypeptide, the term “native” refers to a naturally occurring (e.g., a wild-type (WT)) nucleic acid or polypeptide.

As used herein, the terms “operable linkage” and “operably linked” refer to a physical or functional juxtaposition of the components so described as to permit them to function in their intended manner. In the example of an expression control element in operable linkage with a nucleic acid, the relationship is such that the control element modulates expression of the nucleic acid.

As used herein, a “vector” is a composition of matter which can be used to deliver a nucleic acid of interest to the interior of a cell, including a nucleic acid molecule capable of transporting another nucleic acid to which it has been linked. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adeno-associated virus (AAV) vectors, retroviral vectors, lentiviral vectors, adenoviral vectors, and the like. An expression construct can be replicated in a living cell, or it can be made synthetically. Vectors capable of directing the expression of genes to which they are operatively linked are often referred to as “expression vectors.”

“Purified,” as used herein, means separated from many other compounds or entities. A compound or entity (e.g., nucleic acid, protein, virus, viral vector) may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%.

The phrases “isolated” or “biologically pure” refer to material which is substantially or essentially free from components which normally accompany it as found in its native state.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der waals).

The term “labeled,” with regard to a nucleic acid, nanoparticle, virus, peptide, polypeptide, cell, probe or antibody, is intended to encompass direct labeling of the nucleic acid, nanoparticle, virus, peptide, polypeptide, cell, probe or antibody by coupling (i.e., physically linking) a detectable substance to the nucleic acid, nanoparticle, virus, peptide, polypeptide, cell, probe or antibody.

As used herein, the term “therapeutic agent” is meant to encompass any molecule, chemical entity, composition, recombinant virus, nanoparticle, nucleic acid, drug, or biological agent capable of curing, healing, alleviating, relieving, altering, remedying, ameliorating, improving or affecting a disease, the symptoms of disease, or the predisposition toward disease. The term “therapeutic agent” includes natural or synthetic compounds, molecules, chemical entities, compositions, recombinant viruses, nanoparticles, nucleic acids, etc.

As used herein, the terms “treatment” and “therapy” are defined as the application or administration of a therapeutic agent to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease. Methods and uses of the compositions, nanoparticles, vectors, and viruses described herein include treatment methods, which result in any therapeutic or beneficial effect. In particular aspects of the methods and uses of the compositions, nanoparticles, vectors, and viruses disclosed herein, expression of a nucleic acid encoding pre-miR-7 provides a therapeutic benefit to the mammal (e.g., human suffering from SCI). In various embodiments, further included are inhibiting, decreasing, or reducing one or more adverse (e.g., physical) symptoms, disorders, illnesses, diseases, or complications caused by or associated with a disease (e.g., impaired locomotor function).

By the phrases “therapeutically effective amount” and “effective dosage” is meant an amount sufficient to produce a therapeutically (e.g., clinically) desirable result; for example, the result can be increasing (promoting) neuronal survival and axon regeneration in a subject, improving locomotor function and/or bladder function and/bowel function, and/or alleviating numbness or tingling, in a subject, and treating SCI in a subject (e.g., mammals including humans).

As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two RNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity can be measured using any appropriate sequence analysis software.

Although compositions, nanoparticles, vectors, viruses, kits, and methods similar or equivalent to those described herein can be used in the practice or testing of the present disclosure, suitable compositions, nanoparticles, vectors, viruses, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

miRNA and Compositions thereof

MicroRNAs (miRNAs) are a class of endogenous 17-24 base-long single-stranded, non-coding RNAs that regulate gene expression in a sequence-specific manner in plants and animals. miRNAs are encoded within the genome as independent genomic transcription units or as introns of protein-coding genes and require multiple processing steps that take place in both the nucleus and the cytoplasm. miRNAs are initially transcribed in the nucleus by the RNA polymerase II as long primary miRNAs (pri-miRNAs). The pri-miRNAs are then converted by the RNAse III enzyme Drosha into 60 bp stem-loop structures called pre-miRNAs. The pre-miRNAs are subsequently exported to the cytoplasm by Exportin 5 via a nuclear pore.

Once in the cytoplasm, the pre-miRNAs are further processed by a second RNase II enzyme called Dicer into a 21-22 nucleotide miRNA-miRNA complex intermediate. This RNA-duplex then binds to an Argonaute (AGO) protein. Importantly, unlike small interfering RNA (siRNA), double-stranded miRNA duplexes have mismatches that allow unwinding of the duplex and removal of one of the strands after binding, resulting in the mature RNA-induced silencing complex (RISC). The strand fully bound to RISC is used as a template to find mRNA targets, with most miRNAs having selectivity toward the 3′ untranslated region (UTR) of the target mRNA. Target recognition is typically determined by base-pairing between the seed sequence of the miRNA strand, which is approximately 2-8 nucleotides from the 5′ end, and the mRNA transcript.

Once a target mRNA is found, RISC will bind to complementary mRNA sequences and repress their expression by: (i) translational repression via blocking translational initiation, poly(A) tail shortening, or recruiting translation blockers; (ii) mRNA decay; or (iii) direct cleavage of target mRNAs by RISC. Because only the short seed sequence is responsible for target binding, a single miRNA can target many sequences thereby regulating the expression of multiple genes.

miRNAs have been suggested to play important roles in diverse biological phenomena including development, oncogenesis, and brain functions. Some miRNAs are specifically expressed and enriched in the brain, and have been associated with memory, neuronal differentiation and synaptic plasticity. Several studies have implicated miRNAs in brain diseases. For example, a mutation in the target site of miR-189 in the human SLITRK1 gene has been shown to be associated with Tourette's syndrome. In addition, conditional deletion of Dicer in murine post-mitotic Purkinje cells resulted in progressive loss of miRNAs, cerebellar degeneration and ataxia. miRNAs regulate the expression of ataxin1, amyloid precursor protein (APP) and BACE1, and have been suggested to contribute to neurodegenerative disorders. Interestingly, miR-133b, which is specifically expressed in midbrain dopaminergic neurons and controls their maturation and function through its effect on the homeodomain transcription factor Pitx3, is deficient in PD brains, suggesting that miR-133b is essential for the maintenance of these neurons and could therefore play a role in PD pathogenesis.

Additionally, miR-7, which is a brain-enriched miRNA, binds to the 3′UTR of α-Syn mRNA in a sequence dependent manner and significantly inhibits its translation. GenBank BLAST search revealed that miR-7 is found in human, mouse, rat, zebra fish and fly, suggesting that it regulates biological functions conserved between vertebrates and invertebrate. Anti-sense inhibition of miR-7 has been found to down-regulate cell growth and increase apoptosis, suggesting that miR-7 has a protective role. The latter finding is consistent with observation that miR-7 suppresses α-Syn mediated cell death. In contrast, miR-7 can also have tumor suppressor-like characteristics in glioblastomas. It potently down-regulates the EGF receptor (EGFR) as well as upstream players of the Akt pathway. Additionally, miR-7 is down-regulated in human glioblastoma tissue relative to surrounding normal brain. The apparent discrepancy between the anti- and pro-cell death activity of miR-7 might reflect the complex regulatory role of this microRNA, requiring additional investigations into its biology in different cellular contexts.

miR-7 is transcribed from three loci in the human genome and one locus of the mouse genome. miR-7-1 is located within an intron of the HNRNPK gene on chromosome 9, which encodes a ribonucleoprotein. Profiling microRNA expression in various tissues has found miR-7 highly expressed in the pituitary gland, presumably because miR-7-3 is transcribed from an intron of pituitary gland-specific factor 1a (PGSF1) gene.

Based on a prediction algorithm of Target Scan v5.1, miRNA-153, miRNA-223, miRNA-504, miRNA-920, miRNA-34b, miRNA-374, miRNA-129, miRNA-144, miRNA-143, miRNA-148, and miRNA-433, based on their similar characteristics to miR-7, may also repress α-Syn expression and inhibit α-Syn-mediated cell death. Some of these target sequences in the 3′-UTR of α-Syn are highly conserved.

Several polymorphisms in the 3′-UTR of the human α-Syn gene are reported in GeneBank but not in the target sites of miR-7 or miR-153. Instead, a polymorphic variation (rs10024743) lies in the potential target site of miR-34b. A polymorphic variation in the miR-433 binding site of the fibroblast growth factor 20 (FGF20) gene was recently reported to confer risk of PD which was attributed to increasing FGF20 levels and indirectly α-Syn expression. These observations raise the possibility that mutations in miR-binding sites or in miR genes themselves can trigger neurodegenerative disease.

There are several challenges to developing miR-based therapeutics. One of them is the biological instability of these compounds in biological fluids or tissues as unmodified oligonucleotides are rapidly degraded by cellular and serum nucleases. Another problem is the poor cellular uptake of oligonucleotides due to their size and negative charge, which could prevent them from crossing through cell membranes. These limitations reduce the effective therapeutic use of miRNAs.

Importantly, unlike siRNA, which has a defined anti-sense strand, either of the two strands of miRNA duplex (usually represented as miR (5p) and miR*(3p)) can participate in gene silencing activity. However, the miR strand typically plays the major role, whereas the miR*strand is often degraded. Selection of the miR or miR*strand within the RISC complex is based on the orientation of the RNA duplex within the protein complex. In general, miRNA loading is based on the thermodynamic asymmetry of the two ends of the duplex. The strand with the less stable 5′ end is more likely to be selected as a “guide” strand; the other strand, with a more stable 5′ end, serves as a “passenger” strand that is ultimately degraded. Unlike with the more readily modifiable design of siRNA, however, optimization of miRNAs has been challenging.

Recent reports of non-chemical modification of miR-34a, involved in several types of cancer, osteoporosis, bone metastasis, and cardiovascular disease, suggest that strand activity within the miRNA duplex can be regulated by the addition of deoxyribonucleotides to the miR (5p) or miR*(3p) strands. Kadekar et al. Molecular Therapy: Nucleic Acids 16:597, 2019. The present disclosure provides novel compositions of miRNA or mimics thereof, including miR-7, miR-153, miR-34b, miR-34c, and miR-155, incorporating non-chemical deoxyribonucleotide modifications for enhanced efficacy in the therapeutic treatment of nervous system dysfunction.

A person of ordinary skill in the art will appreciate that the disclosure is not limited to the miRNA agents comprising any one of SEQ. IDs NO. 1-30 (see Table 1 below) and that other miRNA agents are also suitable for the compositions and methods of the instant disclosure.

TABLE 1
Example wild type and modified miRNA guide and
passenger strand sequences

| | Seq | |
|---|---|---|
| | ID | |
| | No. | Sequence (5′→3′) |
| mir-7-5p | 1 | UGGAAGACUAGUGAUUUUGUUGUU |
| mir-7-5p | 2 | CAACAAAUCACAGUCUGCCAUA |
| mir-7-5p mimic | 3 | CAACAAAUCACAGUCUGCCAUA(dT)₅ |
| mir-7-5p mimic | 4 | CAACAAAUCACAGUCUGCCAUA(dC)₅ |
| mir-7-5p mimic | 5 | CAACAAAUCACAGUCUGCCAUA(dA)₅ |
| mir-7-5p mimic | 6 | CAACAAAUCACAGUCUGCCAUA(dG)₅ |
| mir-153-3p | 7 | UUGCAUAGUCACAAAAGUGAUC |
| mir-153-3p | 8 | UCAUUUUUGUGAUGUUGCAGCU |
| mir-153-3p mimic | 9 | UCAUUUUUGUGAUGUUGCAGCU(dT)₅ |
| mir-153-3p mimic | 10 | UCAUUUUUGUGAUGUUGCAGCU(dC)₅ |
| mir-153-3p mimic | 11 | UCAUUUUUGUGAUGUUGCAGCU(dA)₅ |
| mir-153-3p mimic | 12 | UCAUUUUUGUGAUGUUGCAGCU(dG)₅ |
| mir-34b-3p | 13 | CAAUCACUAACUCCACUGCCAU |
| mir-34b-3p | 14 | UAGGCAGUGUCAUUAGCUGAUUG |
| mir-34b-3p mimic | 15 | UAGGCAGUGUCAUUAGCUGAUUG |
| | | (dT)₅ |
| mir-34b-3p mimic | 16 | UAGGCAGUGUCAUUAGCUGAUUG |
| | | (dC)₅ |
| mir-34b-3p mimic | 17 | UAGGCAGUGUCAUUAGCUGAUUG |
| | | (dA)₅ |
| mir-34b-3p mimic | 18 | UAGGCAGUGUCAUUAGCUGAUUG |
| | | (dG)₅ |
| mir-34c-5p | 19 | AGGCAGUGUAGUUAGCUGAUUGC |
| mir-34c-5p | 20 | AAUCACUAACCACACGGCCAGG |
| mir-34c-5p mimic | 21 | AAUCACUAACCACACGGCCAGG(dT)₅ |
| mir-34c-5p mimic | 22 | AAUCACUAACCACACGGCCAGG(dC)₅ |
| mir-34c-5p mimic | 23 | AAUCACUAACCACACGGCCAGG(dA)₅ |
| mir-34c-5p mimic | 24 | AAUCACUAACCACACGGCCAGG(dG)₅ |
| mir-155-5p | 25 | UUAAUGCUAAUCGUGAUAGGGGUU |
| mir-155-5p | 26 | CUCCUACAUAUUAGCAUUAACA |
| mir-155-5p mimic | 27 | CUCCUACAUAUUAGCAUUAACA(dT)₅ |
| mir-155-5p mimic | 28 | CUCCUACAUAUUAGCAUUAACA(dC)₅ |
| mir-155-5p mimic | 29 | CUCCUACAUAUUAGCAUUAACA(dA)₅ |
| mir-155-5p mimic | 30 | CUCCUACAUAUUAGCAUUAACA(dG)₅ |

In some embodiments, the microRNA or mimic thereof comprises a microRNA selected from the group comprising miRNA-7, miRNA-153, miRNA-34b, miRNA-34c, and miRNA-155.

In some embodiments, the microRNA, or mimic thereof, comprises a first guide nucleotide sequence having at least 80% (e.g., 80%, 85%, 90%, 95%, 99%) identity to SEQ ID NO: 1 or comprising SEQ ID NO: 1.

In some embodiments, the microRNA, or mimic thereof, comprises a second passenger sequence having at least 80% (e.g., 80%, 85%, 90%, 95%, 99%) identity to a nucleic acid sequence selected from SEQ ID NOs: 2-6 or comprising a nucleotide sequence selected from SEQ ID NOs: 2-6.

In some embodiments, the microRNA, or mimic thereof, comprises a first guide nucleotide sequence having at least 80% (e.g., 80%, 85%, 90%, 95%, 99%) identity to SEQ ID NO: 7 or comprising SEQ ID NO: 7.

In some embodiments, the microRNA, or mimic thereof, comprises a second passenger nucleotide sequence having at least 80% (e.g., 80%, 85%, 90%, 95%, 99%) identity to a nucleic acid sequence selected from SEQ ID NOs: 8-12 or comprising a nucleotide sequence selected from SEQ ID NOs: 8-12.

In some embodiments, the microRNA, or mimic thereof, comprises a first guide nucleotide sequence having at least 80% (e.g., 80%, 85%, 90%, 95%, 99%) identity to SEQ ID NO: 13 or comprising SEQ ID NO: 13.

In some embodiments, the microRNA, or mimic thereof, comprises a second passenger nucleotide sequence having at least 80% (e.g., 80%, 85%, 90%, 95%, 99%) identity to a nucleic acid sequence selected from SEQ ID NOs: 14-18 or comprising a nucleotide sequence selected from SEQ ID NOs: 14-18.

In some embodiments, the microRNA, or mimic thereof, comprises a first guide nucleotide sequence having at least 80% (e.g., 80%, 85%, 90%, 95%, 99%) identity to SEQ ID NO: 19 or comprising SEQ ID NO: 19.

In some embodiments, the microRNA, or mimic thereof, comprises a second passenger nucleotide sequence having at least 80% (e.g., 80%, 85%, 90%, 95%, 99%) identity to a nucleic acid sequence selected from SEQ ID NOs: 20-24 or comprising a nucleotide sequence selected from SEQ ID NOs: 20-24.

In some embodiments, the microRNA, or mimic thereof, comprises a first guide nucleotide sequence having at least 80% (e.g., 80%, 85%, 90%, 95%, 99%) identity to SEQ ID NO: 25 or comprising SEQ ID NO: 25.

In some embodiments, the microRNA, or mimic thereof, comprises a second passenger nucleotide sequence having at least 80% (e.g., 80%, 85%, 90%, 95%, 99%) identity to a nucleotide sequence selected from SEQ ID NOs: 26-30 or comprising a nucleotide sequence selected from SEQ ID NOs: 26-30.

In some embodiments, the above-described agents and the delivery system may be incorporated into a composition (including inhalation, intradermal, transdermal, parenteral, intravenous, intramuscular, intranasal, subcutaneous, percutaneous, intratracheal, intraventricular, intra-brain tissue, intra-spinal cord, intraperitoneal, intra-tumoral, perfusion, lavage, oral, or direct injection preparations). In some embodiments, the composition may include from about 0.00001 to 100%, such as from 0.001 to 10% or from 0.1% to 5% by weight of one or more agents described herein.

In some embodiments, a disclosed agent (e.g., a nucleic acid therapeutic) described herein may be incorporated into a topical formulation containing a topical carrier that is generally suited to topical drug administration and comprising any such material known in the art. The topical carrier may be selected to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols, and other nontoxic organic solvents, glycerin, mineral oil, silicone, petroleum jelly, lanolin, fatty acids, vegetable oils, parabens, waxes, and the like.

In some embodiments, the compositions may be pharmaceutically acceptable or pharmacologically acceptable compositions. Pharmaceutically acceptable carriers are determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, liquid polyethylene glycol, and the like), and suitable mixtures thereof. The proper fluidity can be maintained, e.g., by the use of a coating such as lecithin, by the maintenance of the required particle size in the case of dispersion, and by the use of surfactants Typically, such compositions are prepared either as topical compositions, liquid solutions or suspensions, solid forms suitable for solution in, or suspension in, liquid prior to use can also be prepared. Routes of administration can vary with the location and nature of the condition to be treated, and include, e.g., topical, inhalation, intradermal, transdermal, parenteral, intravenous, intramuscular, intranasal, subcutaneous, percutaneous, intratracheal, intravenous, intraventricular, intra-brain tissue, intra-spinal cord, intraperitoneal, intra-tumoral, perfusion, lavage, oral, or direct injection administration and formulation.

In some embodiments, the disclosed pharmaceutical compositions can also include adjuvants such as aluminum salts and other mineral adjuvants, tensioactive agents, bacterial derivatives, vehicles, and cytokines. Adjuvants can also have antagonizing immunomodulating properties. For example, adjuvants can stimulate Th1 or Th2 immunity. In some embodiments, the pharmaceutical compositions can be formulated in any conventional manner using one or more physiologically acceptable carriers and/or excipients.

In some embodiments, the pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. In some embodiments, the pharmaceutical compositions can further be formulated as suspensions, solutions, or emulsions in oily or aqueous vehicles and may contain other agents, including suspending, stabilizing and/or dispersing agents.

In some embodiments, the pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A carrier can also be a solvent or dispersion medium containing, for example, water, saline, polyol (for example, glycerol, propylene glycol, liquid polyethylene glycol, and the like), suitable mixtures thereof, and vegetable oils. The prevention of the action of microorganisms can be brought about by various antibacterial and antifungal agents known in the art. In many cases, it will be preferable to include isotonic agents, for example, sugars or sodium chloride.

In some embodiments, compositions described herein may include a therapeutically effective amount of a nucleic acid sequence encoding a pre-miRNA. In some embodiments, the nucleic acid sequence encoding pre-miRNA is complexed with a nanoparticle (e.g., a gold nanoparticle) and PEG. In other embodiments, the nucleic acid sequence encoding pre-miRNA is included within a gene therapy vector (a gene therapy vector including a polynucleotide sequence including a nucleic acid sequence encoding pre-miRNA). The compositions can also include a pharmaceutically acceptable carrier.

In some embodiments in which a nucleic acid sequence encoding pre-miRNA is included within a gene therapy vector, it is typically contained with a viral vector. The vectors may be episomal, or may be integrated into the target cell genome, through homologous recombination or random integration. Any suitable viral vector can be used. Viruses are naturally evolved vehicles which efficiently deliver their genes into host cells and therefore are desirable vector systems for the delivery of therapeutic nucleic acids. Preferred viral vectors exhibit low toxicity to the host cell and produce/deliver therapeutic quantities of the nucleic acid of interest (in some embodiments, in a tissue-specific manner). A number of viral vector-based systems have been developed for gene transfer into mammalian cells. For example, AAV provide a convenient platform for gene delivery systems. As another example, retroviruses provide a convenient platform for gene delivery systems. In yet other examples, adenovirus vectors, retrovirus vectors, herpesvirus vectors, alphavirus vectors, or lentivirus vectors are used. A selected nucleic acid sequence can be inserted into a vector (a vector genome) and packaged in viral particles using techniques known in the art (e.g., an rAAV vector packaged in rAAV particles, Vesicular Stomatitis Virus (VSV) G-pseudotyped lentivirus, etc.). The recombinant virus can then be isolated and delivered to cells of the subject.

In some embodiments, an AAV serotype having spinal cord tissue tropism is used. For example, in humans, AAV1 has shown widespread transduction ability and long-lasting gene expression (for reviews of in vivo tissue tropisms, see Nonnenmacher M. and Weber T. (2012) Gene Ther. 19:649-658; Agbandje-McKenna M. and Kleinschmidt J. (2011) AAV capsid and cell interactions—In Adeno-Associated Virus: Methods and Protocols, ed. RO Snyder, P Moullier, p. 47-92, Humana Press, Clifton, NJ; and Asokan A. et al. (2012) Mol. Ther. 4:699-708). In some embodiments, rAAV with serotype 9 capsid proteins is used because rAAV9 has become a preferred vector for CNS delivery due to its increased ability to cross the blood-brain barrier (Lukashchuk V et al. Molecular therapy-Methods and clinical development 3:15055, 2016). In some embodiments, AAV-PHP.eB is used as a vector due to its increased ability to cross the blood-brain barrier (Mathieson S. et al., Molecular therapy—Methods and clinical development 19:447 (2020).

Methods are well known in the art for generating rAAV vectors and rAAV (virions) having improved features for delivering therapeutic agents. rAAV having new capsid variants that, for example, have higher transduction frequency or increased spinal cord tissue tropism, can be used. For example, capsid libraries can be screened in a process called directed evolution (Bartel M. A. (2012) Gene Ther. 19:694-700) to select capsids enriched for infecting a particular tissue or cell type. As another example, rAAV having capsids decorated with ligand targeted to a specific cell type (e.g., spinal cord tissue-specific) can be used. As another example, pseudotyped (also referred to as transcapsidated) rAAV (nucleic acid or genome derived from a first AAV serotype that is encapsidated or packaged by an AAV capsid containing at least one AAV Cap protein of a second serotype (i.e., one different from the first AAV serotype)) can be used. rAAV having mosaic capsids are packaged with a mixture of capsid proteins from two different serotypes. In addition to capsid modifications, rAAV as described herein may include tissue-specific promoters (e.g., spinal cord-specific promoters) and inducible promoters. For a review of rAAV gene therapy methods, see Samulski, R. J. and Muzyczka, N. (2014) AAV-Mediated Gene Therapy for Research and Therapeutic Purposes, Annu. Rev. Virol. 1:427-451. rAAV, variants, chimeras, and rAAV-mediated gene transfer methods are also described in U.S. Pat. No. 9,840,719.

rAAV can be produced using any suitable methods. Methods for large-scale production of rAAV are known and are described in Urabe M. J. (2006) Virol. 80:1874-1885; Kotin R. M. (2011) Hum. Mol. Genet. 20:R2-6; Kohlbrenner E. et al. (2005) Mol. Ther. 12:1217-1225; Mietzsch M. (2014) Hum. Gene Ther. 25:212-222; and U.S. Pat. Nos. 6,436,392, 7,241,447, and 8,236,557.

Construction, large-scale manufacturing, and clinical use of third-generation SIN lentiviral vectors are well known in the art. See, for example, Ghani et al. Mol Ther Methods Clin Dev. 2019 Sep. 13; 14: 90-99; and Hu et al. Mol Ther Methods Clin Dev. 2015; 2: 15004.

The viral vectors described herein typically include one or more expression control elements. Expression control elements include ubiquitous or promiscuous promoters/enhancers which are capable of driving expression of a polynucleotide (nucleic acid) in many different cell types. Such elements include, but are not limited to the EF 1a promoter, the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature, the SV40 promoter, the dihydrofolate reductase (DHFR) promoter, the cytoplasmic β-actin promoter, the phosphoglycerol kinase (PGK) promoter, etc.

Expression control elements include those active in a particular tissue or cell type, referred to herein as a “tissue-specific expression control elements/promoters.” Tissue-specific expression control elements are typically active in a specific cell or tissue (e.g., spinal cord). Expression control elements also can confer expression in a manner that is regulatable, that is, a signal or stimuli increases or decreases expression of the operably linked nucleic acid. A regulatable element that increases expression of the operably linked nucleic acid in response to a signal or stimuli is also referred to as an “inducible element” (i.e., is induced by a signal). A regulatable element that decreases expression of the operably linked nucleic acid in response to a signal or stimuli is referred to as a “repressible element” (i.e., the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression.

Expression control elements also include native elements(s). A native control element (e.g., promoter) may be used when it is desired that expression of the nucleic acid may mimic the native expression. A native element may be used when expression of the nucleic acid is to be regulated temporally or developmentally, or in a tissue-specific manner, or in response to specific transcriptional stimuli. Other native expression control elements, such as introns, polyadenylation sites or Kozak consensus sequences may also be used.

As indicated above, in some embodiments, a composition for improving locomotor function in a subject having a SCI includes a nanoparticle complexed with PEG and a nucleic acid sequence encoding pre-miRNA. In general, nanoparticles contemplated include any compound or substance with a high loading capacity for a nucleic acid (e.g., pre-miR-7) as described herein, including for example and without limitation, a metal, a semiconductor, and an insulator particle composition, and a dendrimer (organic versus inorganic). Thus, nanoparticles are contemplated which include a variety of inorganic materials including, but not limited to, metals, semi-conductor materials or ceramics. In one embodiment, the nanoparticle is metallic, and in various aspects, the nanoparticle is a colloidal metal. Thus, in various embodiments, nanoparticles of the disclosure include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Nanoparticles as described herein include those that are available commercially (e.g., Nanohybrids), as well as those that are synthesized, e.g., produced from progressive nucleation in solution (e.g., by colloid reaction) or by various physical and chemical vapor deposition processes. Methods of making metal, semiconductor and magnetic nanoparticles are well-known in the art. Nanoparticles such as gold nanoparticles can be produced using any suitable methods, e.g., those described in Papastefanaki et al. Mol Ther 23:993-1002, 2015; Kao et al. Nanotechnology 25:295102, 2015; Gerard et al. Pain 156:1320-1333, 2015; Bonoiu et al. Proc Natl Acad Scie USA 106:5546-5550; Schmid, G. (ed.) Clusters and Colloids (VCH, Weinheim, 1994); Hayat, M. A. (ed.) Colloidal Gold: Principles, Methods, and Applications (Academic Press, San Diego, 1991); Burda et al., Chem. Rev. 105: 1025-1102, 2005; Daniel and Astruc Chem. Rev. 104: 293-346, 2004′ and U.S. Pat. Nos. 10,391,116, 10,370,661 and 9,382,346.

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. Typically, the size of the nanoparticles is from about 5 nm to about 150 nm (mean diameter), from about 30 to about 100 nm, from about 40 to about 80 nm. In some embodiments, the nanoparticle is optionally labeled. In some embodiments, the nanoparticle further includes a targeting molecule.

In some embodiments, the composition may be incorporated into a kit or a device (e.g., an applicator). In some embodiments, the composition may be incorporated into a medical device, such as an implantable medical device. The above medical devices can be surface modified, for example, plasma treatment or coating with another material, to enhance the affinity of the medical device with nucleic acids formulations.

In another aspect, this disclosure provides a kit or device comprising the composition as described herein. The kit may further include water and hybridization buffer to facilitate hybridization of the two strands of the miRNAs.

Methods of Use

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; The Condensed Protocols From Molecular Cloning: A Laboratory Manual, by Joseph Sambrook et al., Cold Spring Harbor Laboratory Press, Cold Spring Harbor, N.Y., 2006; and Current Protocols in Molecular Biology, ed. Ausubel et al., Greene Publishing and Wiley-Interscience, New York, 1995 (with periodic updates). Conventional methods of gene transfer and gene therapy may also be adapted for use in the present disclosure. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Methods for constructing and using viral vectors are known in the art (see, e.g., Miller and Rosman, BioTechniques 1992, 7:980-990). Methods for large-scale production of rAAV are described in Urabe M. J. (2006) Virol. 80:1874-1885; Kotin R. M. (2011) Hum. Mol. Genet. 20:R2-6; Kohlbrenner E. et al. (2005) Mol. Ther. 12:1217-1225; and Mietzsch M. (2014) Hum. Gene Ther. 25:212-222. For a review of rAAV gene therapy methods, see J. L. Santiago-Ortiz and D. V. Schaffer J Control Release 240:287-301, 2016; Rodrigues et al., Pharm Res 36:29, 2019; Choi et al., Curr Gene Ther 5:299-310, 2005; Samulski, R. J. and Muzyczka, N. (2014) AAV-Mediated Gene Therapy for Research and Therapeutic Purposes, Annu. Rev. Virol. 1:427-451. rAAV vectors, variants, chimeras, and rAAV vector mediated gene transfer methods are described in U.S. Pat. No. 9,840,719. Construction, large-scale manufacturing, and clinical use of third-generation SIN lentiviral vectors are well known in the art.

The miRNA sequence that can be used as an active ingredient of the composition for treating nervous system dysfunction provided by the present disclosure is a sequence derived from the human genome. However, a miRNA sequence derived from the genome of another animal can also be used without limiting the genome of the miRNA.

The miRNA can be used in the form of various miRNA derivatives (miRNA mimics) that generate the bioequivalence efficacy of miRNAs and can be modified miRNAs containing miRNA sequences containing the same seed region. At this time, the length of either miRNA strand can be reduced, and a short derivative consisting of about 12-15 nucleotides can also be used.

The miRNA can be used in the form of precursor miRNA or primary miRNA (pri-miRNA) and can be synthesized by a chemical method or delivered in the form of a plasmid to cells which express the same. According to the present disclosure, methods for delivering miRNAs to cells cultured on culture dishes include, but are not limited to, mixing with cationic lipids, using electrical stimulation, and using viruses.

For purposes of illustration only, Parkinson's Disease (PD) will be discussed further herein. α-Synuclein (α-Syn) is a key player in the pathogenesis of PD based on genetic, neuropathologic, and cellular/molecular lines of evidence. In addition to point mutations linked to dominantly inherited PD, mounting evidence suggests that elevated levels of α-Syn are deleterious to dopaminergic neurons. Individuals with multiplication of this gene locus develop PD with an earlier onset age and increasing severity associated with dementia in a gene dosage dependent manner. Transgenic mice, drosophila, and C. elegans over-expressing α-Syn manifest phenotypic changes reminiscent of the disease, and engineered cultured cells are made vulnerable by this protein. In addition, a large-scale analysis in patients with PD and controls showed that variability in the α-Syn promoter region, which results in its up-regulation, is associated with an increased risk of PD. Besides these compelling data, postmortem investigations of PD and other α-synucleinopathies have demonstrated fibrillar α-Syn aggregates in Lewy bodies and Lewy neurites. An immunization approach to clear the brain of the α-Syn burden has been shown to reduce the neurodegeneration in transgenic mice. Based on the aforementioned evidence, α-Syn over-expression appears to be a common mechanism for the pathogenesis of PD and other α-synucleinopathies.

Increased α-Syn gene (SNCA) dosage due to locus multiplication leads to autosomal dominantly inherited PD, suggesting that higher concentration of α-Syn protein in neurons is involved in the pathogenesis of PD and PD Dementia. In sporadic PD, it is conceivable that various genetic or environmental factors that up-regulate α-Syn expression can be potential culprits as well. Recent evidence suggests that miRNAs regulate a plethora of genes and are involved in many disease states, ranging from cancer to neurodegeneration.

Kits

In some embodiments, the above-described miRNA or the composition (e.g., the pharmaceutical composition) can be provided in a kit. In one embodiment, the kit includes a container that contains the miRNA or the composition, and optionally informational material. The informational material can be descriptive, instructional, marketing, or other material that relates to the methods described herein and/or the use of the agents for therapeutic benefit. For example, kits may include instruction for the manufacturing, for the therapeutic regimen to be used, and periods of administration. In an embodiment, the kit also includes an additional therapeutic agent (e.g., a checkpoint modulator, a chemotherapeutic compound). The kit may comprise one or more containers, each with a different reagent. For example, the kit includes a first container that contains the immune cells or the composition and a second container for the additional therapeutic agent.

In some embodiments, the containers can include a unit dosage of the pharmaceutical composition. In addition to the composition, the kit can include other ingredients, such as a solvent or buffer, an adjuvant, a stabilizer, or a preservative.

In some embodiments, the kit optionally includes a device suitable for administration of the composition, e.g., a syringe or other suitable delivery device. The device can be provided pre-loaded with one or both of the agents or can be empty but suitable for loading.

In some embodiments, the miRNA or the composition, as described herein, may be administered with an additional therapeutic agent or therapy. In some embodiments, the composition can be administered to a subject either simultaneously with, before (e.g., 1-30 days before) or after (e.g., 1-30 days after) the additional therapeutic (including but not limited to small molecules, antibodies, or cellular reagents) that acts to elicit a biological response (e.g., to treat nervous system dysfunction) in the subject. When co-administered with an additional therapeutic, the composition and the additional therapeutic agent may be administered simultaneously or sequentially (in any order). Suitable therapeutically effective dosages for each agent may be lowered due to the additive action or synergy.

The pharmaceutical compositions, as described, can be administered in a manner appropriate to the disease to be treated or prevented. In some embodiments, the miRNA, or mimic thereof, the pharmaceutical composition, the viral vector or viral particle, or nanoparticle is administered topically, inhalationally, intradermally, transdermally, parenterally, intravenously, intramuscularly, intranasally, subcutaneously, percutaneously, intratracheally, intraventricularly, intra-brain tissue, intra-spinal cord, intraperitoneally, intra-tumorally, by perfusion, by lavage, orally, or by direct injection.

The amount and frequency of administration will be determined by factors such as the condition of the patient, and the type and severity of the patient's disease, although appropriate dosages can be determined by clinical trials.

When “a therapeutically effective amount,” “an effective quantity,” or “an effective amount” is indicated, the precise amount of the compositions of the present disclosure to be administered can be determined by a physician having account for individual differences in age, weight, extent of injury, and patient's condition. It can generally be stated that a pharmaceutical composition comprising the miRNA described herein can be administered at a dose of 0.01 mg/kg to 10 g/kg body weight (e.g., 0.01 mg/kg, 0.1 mg/kg, 1 mg/kg, 10 mg/kg, 100 mg/kg, 1 g/kg, 10 g/kg) body weight, including all values and integers within these intervals. The miRNA compositions can also be administered several times at these dosages. The miRNA can be administered using infusion techniques that are commonly known (see, for example, Rosenberg et al., New Eng. J. of Med. 319: 1676, 1988). The optimal dose and treatment regimen for a particular patient can be readily determined by one skilled in the art of medicine by monitoring the patient for signs of the disease and adjusting the treatment accordingly.

In some embodiments, the composition can be administered to the subject in a manner compatible with the dosage formulation and in such amount as is therapeutically effective. Dose ranges and frequency of administration can vary depending on, e.g., the nature of the miRNA produced by the methods described herein and the medical condition as well as parameters of a specific patient and the route of administration used.

Described herein are methods for promoting neurologic recovery, including improving locomotor function, in a subject following SCI. These methods include administering to the subject having SCI an effective amount of a recombinant virus that includes a recombinant viral vector that contains a polynucleotide sequence including a nucleic acid sequence encoding pre-miRNA, or an effective amount of a composition including the recombinant virus. In other embodiments, these methods include administering to the subject a nanoparticle complexed with PEG and a nucleic acid sequence encoding pre-miRNA (e.g., administering a gold nanoparticle to a human). Typically, the compositions, nanoparticles, gene therapy vectors and recombinant viruses are delivered to appropriate target cells in the subject (e.g., human patient). A typical target cell is any neuron, glial cell, or oligodendrocyte.

In some embodiments of a method for promoting functional neurologic recovery in a subject following SCI, a rAAV including a rAAV vector including a polynucleotide sequence including a nucleic acid sequence encoding a pre-miRNA is administered to the subject in a therapeutically effective amount for improving neurologic function. In such embodiments, the rAAV can include serotype 1 or 9 capsid proteins and the rAAV vector can be serotype 2. In other embodiments, the vector can include AAV-PHP.eB. In other embodiments of a method for promoting functional recovery in a subject following SCI, a lentivirus system including a recombinant lentiviral vector that includes a polynucleotide sequence including a nucleic acid sequence encoding a pre-miRNA is administered to the subject in a therapeutically effective amount for improving neurologic function.

Typically, the recombinant virus is administered to the subject at one of the following time points: within 1 hour of SCI injury, within 2 hours of SCI injury, within 4 hours of SCI injury, within 6 hours of SCI injury, within 8 hours of SCI injury, within 12 hours of SCI injury, within 24 hours of SCI injury, within 48 hours of SCI injury, within 72 hours of SCI injury, within 7 days of SCI injury, and within one month of SCI injury. In some embodiments, a single administration is sufficient for promoting functional recovery in a subject following SCI, as the spinal cord cells are transduced with a viral vector, and the vector expresses itself on an ongoing (e.g., long-term) basis. In some embodiments in which the composition, nanoparticle, gene therapy vector or recombinant virus is directly injected into the subject's spinal cord, two or more (multiple) administrations at two or more time points (e.g., over weeks, over months) are performed.

The methods include administration of any of the compositions, nanoparticles, gene therapy vectors and recombinant viruses described herein. Administration of a composition, nanoparticle, vector, or virus as described herein to a subject having SCI results in one or more of: increased neuronal survival, increased axon regeneration, improved bladder function, improved locomotor function, improved bowel function, and alleviating numbness and/or tingling, in the subject. The methods can further include evaluating one or more of locomotor function, bladder function, bowel function, numbness, and tingling in the subject at a time point subsequent to administration of the composition, nanoparticle, gene therapy vector, or recombinant virus.

Combination therapies may be used to improve locomotor function and treat SCI in a subject. In some embodiments, a combination therapy involves administering a composition including a nucleic acid sequence encoding a pre-miRNA (e.g., nanoparticle composition or gene therapy vector as described herein) and a second SCI therapeutic. In such an embodiment, the composition and the second SCI therapeutic can be administered in the same composition simultaneously, or they can be administered at different time points (e.g., two different compositions administered at two different time points). In any combination therapy, the two or more therapeutics can be administered simultaneously, concurrently or sequentially, e.g., at two or more different time points. Typically, such a combination therapy increases neuronal survival and axon regeneration and improves bladder function, bowel function and locomotor function, and alleviates numbness and/or tingling in the subject. In one embodiment of combination therapy, a composition including a nucleic acid sequence encoding a pre-miRNA and a second SCI therapeutic are admixed in the same injection or infusion volume.

Any suitable methods of administering such a composition to a subject may be used. In these methods, the compositions can be administered to a subject by any suitable route, e.g., injection directly into the target site (e.g., spinal cord), intravenous (IV) administration, etc. The compositions may be administered by catheter to a site accessible by a blood vessel. If administered via intravenous injection, the compositions, nanoparticles, vectors, and viruses may be administered in a single bolus, multiple injections, or by continuous infusion (e.g., intravenously, pump infusion). For parenteral administration, the compositions are preferably formulated in a sterilized pyrogen-free form.

The compositions described herein may be in a form suitable for sterile injection. To prepare such a composition, the suitable active therapeutic(s) (e.g., a nucleic acid encoding a pre-miRNA (e.g., pre-miR-7), a vector encoding same, a recombinant virus, a nanoparticle complexed with a nucleic acid encoding pre-miRNA) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the therapeutics is only sparingly or slightly soluble in water, a dissolution enhancing or solubilizing agent can be added, or the solvent may include 10-60% w/w of propylene glycol or the like. The compositions, viruses and viral vectors described herein may be administered to mammals (e.g., rodents, humans, nonhuman primates, canines, felines, ovines, porcines, and bovines) in any suitable formulation according to conventional pharmaceutical practice (see, e.g., Remington: The Science and Practice of Pharmacy (20th ed.), ed. A. R. Gennaro, Lippincott Williams & Wilkins, (2000) and Encyclopedia of Pharmaceutical Technology, eds. J. Swarbrick and J. C. Boylan, Marcel Dekker, New York (1988-1999), a standard text in this field, and in USP/NF). A description of exemplary pharmaceutically acceptable carriers and diluents, as well as pharmaceutical formulations, can be found in Remington: supra. Other substances may be added to the compositions to stabilize and/or preserve the compositions.

A “unit dosage form” as used herein refers to physically discrete units suited as unitary dosages for the subject to be treated; each unit containing a predetermined quantity optionally in association with a pharmaceutical carrier (excipient, diluent, vehicle, or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Viral vectors (e.g., AAV vectors), viruses, nanoparticles, and pharmaceutical compositions thereof, can be packaged in single or multiple unit dosage form for ease of administration and uniformity of dosage.

Effective Doses

The compositions described herein are preferably administered to a mammal (e.g., human) in an effective amount, that is, an amount capable of producing a desirable result in a treated mammal (e.g., increasing neuronal survival and axon regeneration, improving bladder function, bowel function, and locomotor function, reducing tremor, alleviating incoordination, alleviating numbness and/or tingling). Such a therapeutically effective amount can be determined according to standard methods. Toxicity and therapeutic efficacy of the compositions utilized in methods of the disclosure can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently. A delivery dose of a composition, nanoparticle, virus, or vector as described herein is determined based on preclinical efficacy and safety. In some embodiments wherein the nanoparticle or gene therapy vector is injected into the subject's spinal cord, a therapeutically effective amount (e.g., an appropriate dose) for a human would be between about 10 μl and 10 ml (e.g., 10 μl, 100 μl, 1 ml, 10 ml). Typically, the range of titer of the viral vector is between about 1×10¹⁰ and 5×10¹⁴ viral genomes/ml, transducing units/ml, or plaque forming units/ml (e.g., 1×10¹⁰, 1×10¹¹, 1×10¹², 1×10¹³, 1×10¹⁴, 1×10¹⁴).

### OTHER EMBODIMENTS

All nucleic acids, nucleic acid names, genes, gene names, and gene products disclosed herein are intended to correspond to homologs from any species for which the compositions, viruses, vectors, nanoparticles, kits, and methods disclosed herein are applicable. Thus, the terms include, but are not limited to, nucleic acids, genes and gene products from humans, mice, dogs, etc. It is understood that when a nucleic acid, gene or gene product from a particular species is disclosed, this disclosure is intended to be exemplary only, and is not to be interpreted as a limitation unless the context in which it appears clearly indicates. Any improvement may be made in part or all of the compositions, viruses, vectors, nanoparticles, kits, and method steps.

Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the phrases “nucleic acid,” “polynucleotide,” “oligonucleotide,” and “nucleic acid molecule” are used interchangeably to refer to a polymer of DNA and/or RNA, which can be single-stranded, double-stranded, or multi-stranded, synthesized or obtained (e.g., isolated and/or purified) from natural sources, which can contain natural, non-natural, and/or altered nucleotides, and which can contain natural, non-natural, and/or altered internucleotide linkages including, but not limited to phosphoroamidate linkages and/or phosphorothioate linkages instead of the phosphodiester found between the nucleotides of an unmodified oligonucleotide.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or mRNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule, or other functional disruption of, or functional addition to, the genetic material of the cell. Exemplary modifications include modifications in the coding region of a heterologous or homologous polypeptide derived from the reference or parent molecule or a functional fragment thereof.

The terms “treat” or “treatment” of a state, disorder or condition include: (1) preventing, delaying, or reducing the incidence and/or likelihood of the appearance of at least one clinical or sub-clinical symptom of the state, disorder, or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression and/or stopping and/or slowing progression of the state, disorder, or condition or at least one of its clinical or sub-clinical symptoms. The benefit to a subject to be treated is either statistically significant or at least perceptible to the patient or to the physician. Thus, the term “treatment” includes preventing a condition from occurring in a patient, particularly when the patient is predisposed to acquiring the condition; reducing and/or inhibiting the condition and/or its development and/or progression; and/or ameliorating and/or reversing the condition. Insofar as some embodiments of the methods of the presently disclosed subject matter are directed to preventing conditions, it is understood that the term “prevent” does not require that the condition be completely thwarted. Rather, as used herein, the term “preventing” refers to the ability of one of ordinary skill in the art to identify a population that is susceptible to the condition, such that administration of the compositions of the presently disclosed subject matter might occur prior to the onset of the condition. The term does not imply that the condition must be completely avoided.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression, slow down and/or stop progression, or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, a viral vector, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder or pathological condition; reducing or preventing the onset of a disease, symptom, disorder or condition; stopping or slowing progression of a disease, symptoms, disorder, or condition; and generally counteracting a disease, symptom, disorder or pathological condition.

Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “pharmaceutical composition” refers to a mixture of at least one compound useful within this disclosure with other chemical components, such as carriers, nanoparticles, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition, or carrier, such as a liquid or solid filler, diluent, excipient, solvent, nanoparticle, or encapsulating material, involved in carrying or transporting an agent within or to the subject such that it may perform its intended function. Typically, such agents are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

“Parenteral” administration of a composition includes, e.g., subcutaneous (s.c.), intravenous (i.v.), intramuscular (i.m.), intradermal (i.d.), transdermal, intranasal, percutaneous, intratracheal, intraventricular, intra-cerebral tissue, intra-spinal cord, intraperitoneal, intra-tumoral, intrasternal, or direct injection, or perfusion, lavage, or infusion techniques.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “I” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the disclosure. This disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contraindicated by context.

### EXAMPLES

Certain embodiments of the disclosure will now be discussed in the following non-limiting examples. These examples are provided for illustration only and should not be construed as limiting the scope of the disclosure in any way.

### Example 1—Neurodegenerative Disease

In vivo validation of the role of specific microRNAs in protecting neurons in the context of neurodegenerative disease

To validate the ability of miR-7 and other microRNAs that have the potential to down-regulate α-synuclein expression in the brain, highly efficient lentiviral or AAV vectors are generated and injected in the substantia nigra or striatum of wild-type mice. The amount of miRNA expression as well as the amount of α-synuclein mRNA and protein expression are measured. This paradigm is employed to assess the ability of this treatment to protect against Parkinson's disease using laboratory animal models.

To analyze effects of miRNA treatment on mitochondrial function, mice are infected with viral vectors expressing miRNA and subsequently treated with various small molecules or other reagents affecting mitochondrial activity and function. Brain tissue samples are dissected quickly and target protein expression levels are assessed. Dopaminergic neuron degeneration is also assessed.

Viral vectors harboring pri-miRNA cDNA sequences (using Sanger miRbase database) are constructed with an AAV vector. Plasmids are constructed that show high expression in neuronal cells. To monitor miRNA expression easily, an insertion of an Internal Ribosome Entry Sequence (IRES)-GFP expression unit downstream of miRNA cDNA is performed, thereby miRNA and GFP will be expressed bicistronically from a single mRNA. Viral vectors are then harvested by standard methods.

In vitro infection of rAAV-miRNA. After generating rAAV-miRNAs, their effectiveness is confirmed in vitro.

rAAV-miRNA gene transfer in vivo. rAAV-miRNAs are injected into mice nervous system tissue using standard procedures known to one of ordinary skill in the art. Effectiveness of gene transfer is measured using standard procedures.

Effect of miRNAs in murine models of nervous system dysfunction. The rAAV-miRNA vectors are placed in mouse models of nervous system dysfunction. Effects on target RNA and protein levels are assessed at different time points. Throughout the proposed studies, statistical significance between control and experimental groups is determined by standard statistical methods.

### Example 2—SCI

miRNA Compositions for Treatment of a Subject Suffering SCI

SCI refers to damage to the spinal cord, which causes permanent changes in strength, sensation and other body functions below the site of the injury. The initial mechanical damage initiates a complex set of secondary molecular events that largely determine the symptoms and disability of the SCI. Diverse cellular mechanisms responsible for this secondary injury mostly depend on changes of specific gene programs.

miRNAs are delivered to nervous system injury sites using a viral vector and a gold nanoparticle, and its effect on locomotor behavior and cellular responses is assessed post-delivery.

Mitochondrial dysfunction contributes to cell death following SCI. In particular, opening of the mitochondrial permeability transition pore (mPTP) has been linked to cell death following SCI. Therefore, promoting mitochondrial health by limiting mPTP formation could have therapeutic value for the treatment of SCI. miRNAs are delivered to injury sites, and their effects on locomotor behavior and cellular response are subsequently assessed.

Nanoparticles are developed as gene delivery vehicles, which are promising since they provide improved oligonucleotide delivery and stability with minimal toxicity in animal models. In particular, gold nanoparticles are employed for drug delivery due to their non-toxic, non-immunoreactive, and biocompatible characteristics. Gold nanoparticles are used to deliver miRNA into severe compressed spinal cord for evaluation as a potential therapeutic drug treatment for spinal cord injury.

In vivo, the effects of viral-mediated delivery of miRNA on functional recovery in a mouse SCI model are investigated. A compression model is effective because compression spares some spinal cord tissue depending on the severity, which is more pertinent to clinical conditions as the spinal cord is hardly completely transected in accidents.

To evaluate whether miR-7 overexpression can improve neurologic behavior after SCI, a set of motor function assays are performed. Biochemical and immunohistological analyses are also used to investigate cellular responses after SCI.

What is claimed is:

1. A microRNA, or a mimic thereof, for the therapeutic treatment of nervous system dysfunction in a patient in need thereof.

2. The microRNA, or mimic thereof, of claim 1, wherein the microRNA, or mimic thereof, comprises a double-stranded oligonucleotide RNA structure further comprising a first guide RNA nucleotide strand and a second passenger RNA nucleotide strand.

3. The microRNA, or mimic thereof, of claim 1, wherein the microRNA, or mimic thereof, contains one or more chemical or nonchemical modifications.

4. The microRNA, or mimic thereof, of claim 3, wherein the nonchemical modifications comprise the addition of one or more deoxyribonucleotides, selected from deoxythymidine (dT), deoxyadenosine (dA), deoxycytidine (dC), and deoxyguanosine.

5. The microRNA, or mimic thereof, of claim 4, wherein the one or more deoxyribonucleotides are added at the 3′ end of one or more of the RNA strands in the microRNA, or mimic thereof.

6. The microRNA, or mimic thereof, of claim 5, wherein the 3′-modified RNA strand is the passenger strand.

7. The microRNA, or mimic thereof, of claim 1, wherein the microRNA, or mimic thereof, comprises a microRNA selected from the group comprising miRNA-7, miRNA-153, miRNA-34b, miRNA-34c, and miRNA-155.

8. The microRNA, or mimic thereof, of claim 1, wherein the microRNA, or mimic thereof, comprises a first guide nucleotide sequence having at least 90% identity to SEQ ID NO: 1 or comprising SEQ ID NO: 1.

9. The microRNA, or mimic thereof, of claim 1, wherein the microRNA, or mimic thereof, comprises a second passenger sequence having at least 90% identity to a nucleic acid sequence selected from SEQ ID NOs: 2-6 or comprising a nucleotide sequence selected from SEQ ID NOs: 2-6.

10. The microRNA, or mimic thereof, of claim 1, wherein the microRNA, or mimic thereof, comprises a first guide nucleotide sequence having at least 90% identity to SEQ ID NO: 7 or comprising SEQ ID NO: 7.

11. The microRNA, or mimic thereof, of claim 1, wherein the microRNA, or mimic thereof, comprises a second passenger nucleotide sequence having at least 90% identity to a nucleic acid sequence selected from SEQ ID NOs: 8-12 or comprising a nucleotide sequence selected from SEQ ID NOs: 8-12.

12. The microRNA, or mimic thereof, of claim 1, wherein the microRNA, or mimic thereof, comprises a first guide nucleotide sequence having at least 90% identity to SEQ ID NO: 13 or comprising SEQ ID NO: 13.

13. The microRNA, or mimic thereof, of claim 1, wherein the microRNA, or mimic thereof, comprises a second passenger nucleotide sequence having at least 90% identity to a nucleic acid sequence selected from SEQ ID NOs: 14-18 or comprising a nucleotide sequence selected from SEQ ID NOs: 14-18.

14. The microRNA, or mimic thereof, of claim 1, wherein the microRNA, or mimic thereof, comprises a first guide nucleotide sequence having at least 90% identity to SEQ ID NO: 19 or comprising SEQ ID NO: 19.

15. The microRNA, or mimic thereof, of claim 1, wherein the microRNA, or mimic thereof, comprises a second passenger nucleotide sequence having at least 90% identity to a nucleic acid sequence selected from SEQ ID NOs: 20-24 or comprising a nucleotide sequence selected from SEQ ID NOs: 20-24.

16. The microRNA, or mimic thereof, of claim 1, wherein the microRNA, or mimic thereof, comprises a first guide nucleotide sequence having at least 90% identity to SEQ ID NO: 25 or comprising SEQ ID NO: 25.

17. The microRNA, or mimic thereof, of claim 1, wherein the microRNA, or mimic thereof, comprises a second passenger nucleotide sequence having at least 90% identity to a nucleotide sequence selected from SEQ ID NOs: 26-30 or comprising a nucleotide sequence selected from SEQ ID NOs: 26-30.

18. A pharmaceutical composition comprising the microRNA, or mimic thereof, of claim 1.

19. A vector or a viral particle comprising the microRNA, or mimic thereof, of claim 1.

20. A nanoparticle comprising the microRNA, or mimic thereof, of claim 1.

21. A method for the treatment of nervous system dysfunction in a patient in need thereof, comprising administering to the patient the microRNA, or mimic thereof, of claim 1.

22. The method of claim 21, wherein the microRNA, or mimic thereof, the pharmaceutical composition, the vector or viral particle, or the nanoparticle is administered to the patient topically, by inhalation, intradermally, transdermally, parenterally, intravenously, intramuscularly, intranasally, subcutaneously, percutaneously, intratracheally, intraventricularly, intra-brain tissue, intra-spinal cord, intraperitoneally, intra-tumorally, by perfusion, by lavage, orally, or by direct injection.

23. The method of claim 21, wherein the nervous system dysfunction results from a neurodegenerative disease associated with α-synuclein pathology.

24. The method of claim 21, wherein the neurodegenerative disease is Parkinson's Disease, Dementia with Lewy Bodies, Parkinson's Disease Dementia, or Multiple System Atrophy.

25. The method of claim 21, wherein the nervous system dysfunction is caused by spinal cord injury (SCI), Traumatic Brain Injury (TBI), Multiple Sclerosis, Optic Neuritis, Transverse Myelitis, Neuromyelitis Optica (NMO), Acute Disseminated Encephalomyelitis, Ischemic Stroke, Amyotrophic Lateral Sclerosis, and Autism Spectrum Disorders (ASD).

26. The method of claim 21, wherein the microRNA, or mimic thereof, is administered to the patient in one or more doses of 0.01 mg/kg to 10 g/kg body weight of the patient, or the vector or viral particle is administered to the patient in one or more doses of 1×10¹⁰ to 5×10¹⁴ viral genomes/ml.

27. The method of claim 21, wherein the treatment further comprises administering a second agent to the patient in need thereof.

28. The method of claim 21, wherein the second agent comprises the microRNA, or mimic thereof, of claim 1, the pharmaceutical composition of claim 19, the vector or viral particle of claim 19, or the nanoparticle of claim 20.
